(19) United States
(12) Patent Application Publication
(10) Pub. No.: US 2004/0044023 A1
(43) Pub. Date: Mar. 4, 2004

(54) Compositions and methods for treating or preventing memory impairment

(76) Inventor: Marc Cantillon, Wayne, PA (US)

Correspondence Address: FLESHNER & KIM, LLP, P.O. BOX 221200, CHANTILLY, VA, 20153, US

(21) Appl. No.: 10/231,361

(22) Filed: Aug. 30, 2002

(51) Int. Cl. A61K 31/55; A61K 31/4745; A61K 31/473; A61K 31/401; A61K 31/366; A61K 31/225

(52) U.S. Cl. 514/292; 514/297; 514/411; 514/214.03; 514/423; 514/460; 514/548

(57) ABSTRACT

The present invention relates to pharmaceutical compositions for treatment or prevention of memory impairment which comprise a cholinesterase inhibitor and at least one other pharmacologically active agent selected from HMG-CoA reductase inhibitors, cholesterol absorption inhibitors, gamma or beta secretase inhibitors, NMDA antagonists, muscarinic receptor agonists and nicotinic receptor agonists. Additionally, the present invention relates to the use of these pharmaceutical compositions to treat or prevent memory impairment in a mammal, such as a human.

### BACKGROUND OF THE INVENTION

1. Field of the Invention

The present invention relates to pharmaceutical compositions useful for treating or preventing memory impairment in a mammal, such as primary or secondary memory impairment, memory impairment associated dementia and age associated memory impairment. Additionally, this invention relates to methods of treating or preventing memory impairment in a mammal by administering to a mammal, such as a human, an effective amount of at least one cholinesterase inhibitor and at least one pharmacologically active agent selected from HMG-CoA reductase inhibitors, gamma or beta secretase inhibitors, N-methyl-D-aspartate (NMDA) antagonists, muscarinic receptor agonists and/or nicotinic receptor agonists.

2. Background of the Related Art

It has been estimated that at least 10% of persons over the age of 60 will eventually suffer severe mental deterioration. A much larger number will experience sufficient cognitive decline to impede their activities. At the other end of the spectrum, more than half of the residents in nursing homes in the United States have been classified as senile. Such mental deterioration includes memory impairment and progression of memory impairment. Owing to the great number of people affected by such disease states, the search for effective methods to treat such conditions is ongoing.

Galanthamine, a tertiary alkaloid, was discovered accidentally in the 1950's by a Bulgarian pharmacologist in the bulbs and flowers of wild Caucasian snowdrops (Galanthus woronowii). It has also been isolated from the common snowdrop (Galanthus nivalis).

Galanthamine is a cholinesterase inhibitor which is active substantially selectively at nicotinic receptor sites while having substantially no effect on muscarinic receptor sites. Galanthamine is capable of passing the blood-brain barrier and presents no severe side effects in therapeutically necessary doses. Galanthamine has been available for over 40 years in Eastern Europe as a curare reversal agent in anesthetic practice.

Statins are known to dramatically lower cholesterol, particularly LDL cholesterol, more effectively than other classes of cholesterol-lowering drugs. They also lower triglycerides and raise protective HDL-cholesterol. In addition, they have been shown to have a number of other possible heart-protective effects. For example, some may lower blood pressure. These drugs have also been found to have a number of other effects that protect against heart disease, like reducing smooth muscle proliferation.

Statins, also know as HMG-CoA reductase inhibitors, block one step in cholesterol synthesis in humans and have been shown to markedly reduce the incidence of heart attacks, strokes and peripheral arterial disease. They also lower rates of death from heart attacks and stroke. Stroke reduction is not typical with cholesterol-lowering drugs, and probably results from some of the benefits of statins (such as raising HDL-cholesterol, which may protect against heart rhythm problems) rather than from LDL-cholesterol reduction. Illustrative examples of statins include atorvastatin, simvastatin, pravastatin, lovastatin, fluvastatin and cerivastatin.

Simvastatin is a lipid-lowering agent that is derived synthetically from a fermentation product of Aspergillus terreus. After oral ingestion, simvastatin, an inactive lactone, is hydrolyzed to the corresponding β-hydroxy acid form. The β-hydroxy acid form is an inhibitor of 3-hydroxy-3-methylglutaryl-coenzyme A (HMG-CoA) reductase. This enzyme catalyzes the conversion of HMG-CoA to mevalonate, which is an early and rate-limiting step in the biosynthesis of cholesterol. Accordingly, simvastatin is used to treat hyperlipidemia.

Cholesterol absorption inhibitors block absorption of cholesterol in the enterocyte cells. The unabsorbed cholesterol is excreted in the feces. Blocking cholesterol absorption in the intestine decreases the uptake of dietary cholesterol. Cholesterol absorption inhibitors do not remain in the liver or enter the systemic circulation in significant amounts and, therefore, typically show minimal toxicity and a reduced potential for drug interactions.

Beta secretase and gamma secretase are enzymes involved in the production of amyloid beta-peptides. Such amyloid peptides are found in extracellular amyloid plaques in the brains of Alzheimer's patients. Beta and gamma secretase inhibitors are thought to reduce the production of amyloid beta-peptides.

In the mammalian nervous system, cholinergic receptors can be divided into two groups: nicotinic receptors and muscarinic receptors, based on the agonist activities of nicotine and muscarine, respectively; muscarinic receptors are subdivided into m1, m2, m3, m4 and m5 subclasses. Cholinergic receptors normally bind the neurotransmitter acetylcholine, thus triggering the opening of ion channels. Acetylcholine mediates a variety of responses in the central nervous system and is thought to play a role in memory function and cognition. Thus, compounds that bind cholinergic receptors may be characterized as either nicotinic receptor agonists or muscarinic receptor agonists, depending upon the type of receptor to which they bind.

N-methyl-D-aspartate (NMDA) antagonists reduce binding of the excitatory amino acids glutamate and aspartate at NMDA receptors. NMDA antagonists are thought to protect against brain damage in neurological disorders such as stroke.

While these various classes of compounds have been used separately to treat the conditions discussed above, they are not believed to have been used in combination therapy. More specifically, they are not believed to have been used in combination therapy to treat or prevent memory impairment in a mammal, such as a human.

### SUMMARY OF THE INVENTION

An object of the present invention is to solve at least the problems and/or disadvantages described above and to provide at least the advantages described hereinafter.

Accordingly, it is an object of the present invention to provide pharmaceutical compositions, as well as methods of using the pharmaceutical compositions, to treat or prevent memory impairment in a mammal, such as a human. Other objects, features and advantages of the present invention will be set forth in the detailed description of preferred embodiments that follows and, in part, will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a pharmaceutical composition comprising: (a) at least one cholinesterase inhibitor; (b) at least one other pharmacologically active agent selected from HMG-CoA reductase inhibitors, cholesterol absorption inhibitors, gamma or beta secretase inhibitors, NMDA antagonists, muscarinic receptor agonists and nicotinic receptor agonists; and (c) a pharmaceutically acceptable carrier.

A second embodiment of the present invention is directed to a method for treating or preventing memory impairment in a mammal in need thereof including administering to a mammal in need thereof a combination of effective amounts of: (a) at least one cholinesterase inhibitor; and (b) at least one other pharmacologically active agent selected from HMG-CoA reductase inhibitors, cholesterol absorption inhibitors, gamma or beta secretase inhibitors, NMDA antagonists, muscarinic receptor agonists and nicotinic receptor agonists.

Additional advantages, objects and feature of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### DETAILED DESCRIPTION OF PREFERRED EMBODIMENTS

A first preferred embodiment of the present invention is directed to a pharmaceutical composition for the treatment or prevention of memory impairment, comprising (a) an effective amount of at least one cholinesterase inhibitor; (b) an effective amount of at least one other pharmacologically active agent selected from the group consisting of HMG-CoA reductase inhibitors, cholesterol absorption inhibitors, gamma or beta secretase inhibitors, NMDA antagonists, muscannic receptor agonists and nicotinic receptor agonists; and (c) a pharmaceutically acceptable carrier.

A second preferred embodiment of the present invention is directed to a method for treating or preventing memory impairment in a mammal in need thereof comprising administering to said mammal in need thereof an effective amount of: (a) at least one cholinesterase inhibitor; and (b) and an effective amount of at least one other pharmacologically active agent selected from the group consisting of HMG-CoA reductase inhibitors, cholesterol absorption inhibitors, gamma or beta secretase inhibitors, NMDA antagonists, muscarinic receptor agonists and nicotinic receptor agonists to treat a patients symptoms of neurological degeneration including memory loss. In certain particularly preferred embodiments of the present invention the mammal is a human.

The pharmaceutical compositions according to the present invention include an effective amount of at least one cholinesterase inhibitor and an effective amount of at least one other pharmacologically active agent. The at least one other pharmacologically active agent is a HMG-CoA reductase inhibitor, a cholesterol-absorption inhibitor, a gamma or beta secretase inhibitor, an NMDA antagonist, a muscarinic receptor agonist, or a nicotinic receptor agonist.

Preferably, the cholinesterase inhibitor is physostigmine, tacrine and tacrine analogues, galanthamine, epigalanthamine, norgalanthamine, fasciculin, metrifonate, heptyl-physostigmine, norpyridostigmine, nomeostigmine or huperzine. More preferably the cholinesterase inhibitor is galanthamine.

In certain preferred embodiments of the present invention, the pharmaceutical composition comprises at least one cholinesterase inhibitor and at least one HMG-CoA reductase inhibitor. According to such embodiments, the HMG-CoA reductase inhibitor is preferably a statin. More preferably the HMG-CoA reductase inhibitor is atorvastatin, simvastatin, pravastatin, lovastatin, fluvastatin or cerivastatin, and most preferably the HMG-CoA reductase inhibitor is simvastatin.

In another preferred embodiment of the present invention, the pharmaceutical composition comprises at least one cholinesterase inhibitor and at least one cholesterol absorption inhibitor. Examples of suitable cholesterol absorption inhibitors include, but are not limited to, substituted azetidinones, such as those disclosed in U.S. Pat. Nos. 5,627,176; 5,631,365; 5,656,624; 5,688,785; and 5,767,115, incorporated herein by reference, and sulfur substituted azetidinones, such as those disclosed in U.S. Pat. Nos. 4,774,467; 5,633,246; and 5,624,920, incorporated herein by reference. In certain particularly preferred embodiments of the present invention, the cholesterol absorption inhibitor is ezetimibe. In even more preferred embodiments of the present invention, the pharmaceutical composition comprises a cholinesterase inhibitor, an HMG-CoA reductase inhibitor and a cholesterol absorption inhibitor.

In other preferred embodiments of the present invention, the pharmaceutical composition comprises at least one cholinesterase inhibitor and at least one muscarinic receptor agonist. According to such embodiments, the muscarinic receptor agonist is preferably aceclide, pilocarpine, oxotremorine, arecaidine, 5-methylfurmethiodide or a pharmaceutically acceptable salts or analog thereof.

In still other preferred embodiments of the present invention, the pharmaceutical composition comprises at least one cholinesterase inhibitor and at least one nicotinic receptor agonist. According to such embodiments, the nicotinic receptor agonist is preferably nicotine or one of its analogs, trans-metanicotine or one of its analogs, epibatidine or one of its analogs, pyridol or derivatives thereof, piperidine alkaloids (such as lobeline and analogs thereof) and imidacloprid or one of its analogs.

In other preferred embodiments of the present invention, the pharmaceutical composition comprises at least one cholinesterase inhibitor and at least one gamma or beta secretase inhibitor. Preferred gamma or beta secretase inhibitors include H-Lys-Thr-Glu-Glu-Ile-Ser-Glu-ValAsn-Sta-Val-Ala-Glu-Phe-OH, presenilin-1, presenilin-2 and derivatives thereof comprising one or more conservative substitutions.

In still other preferred embodiments of the present invention, the pharmaceutical composition comprises at least one cholinesterase inhibitor and at least one NMDA receptor antagonist. Examples of suitable NMDA receptor antagonists include, but are not limited to ketamine, phencyclidine, dizocilpine, tiletamine, dextromethorphan, memantine, amantadine, methadone, dextropropoxyphene, ketobemidone and pharmaceutically acceptable salts thereof.

Additionally, according to certain preferred embodiments of the present invention, the pharmaceutical composition includes, in addition to at least one cholinesterase inhibitor, an effective amount of mixtures of two or more members selected from the group consisting of HMG-CoA reductase inhibitors, cholesterol absorption inhibitors, gamma or beta secretase inhibitors, NMDA receptor antagonists, muscarinic receptor agonists and nicotinic receptor agonists.

Moreover, pharmaceutical compositions according to the present invention may include pharmaceutically acceptable salts of any of the at least one cholinesterase inhibitor and at least one HMG-CoA reductase inhibitor, cholesterol absorption inhibitor, gamma or beta secretase inhibitor, NMDA receptor antagonist, muscarinic receptor agonist and nicotinic receptor agonist.

According to certain preferred embodiments of the present invention, the cholinesterase inhibitor is galanthamine. According to such embodiments, the galanthamine maybe employed in any suitable convenient chemical form known and available to those skilled in the art. For example, in certain embodiments of the present invention, acid addition salts of galanthamine may be used. Illustrative examples of suitable addition salts include, but are not limited to, hydrobromide, hydrochloride, methylsulfate and methiodide salts.

Additionally, according to other preferred embodiments of the present invention, derivatives of galanthamine may be utilized. Illustrative examples of suitable derivatives include, but are not limited to, those disclosed in U.S. Pat. Nos. 6,150,354 and 6,268,358, incorporated herein by reference

In practicing the methods of the present invention, suitable dosages of each pharmacologically active agent can easily be determined empirically by one having ordinary skill in the art. Doses may be varied according to the age, body weight, severity of memory impairment and other conditions known to those skilled in the art.

In certain embodiments of the present invention, the cholinesterase inhibitor is preferably employed in total dosage of up to about 2000 mg per day, more preferably about 5 to about 1000 mg per day and most preferably about 100 to about 600 mg per day.

The other pharmacologically active agent, such as a HMG-CoA reductase inhibitor, is preferably employed in total dosages of up to about 2000 mg per day, more preferably about 10 to about 100 mg per day.

In practicing the methods of the present invention, the total dosage(s) may be given once daily or in multiple dosage forms. Similarly, the pharmacologically active agents maybe taken simultaneously or at spaced time intervals.

When employed in the present methods, the pharmaceutical compositions according to the present invention may be administered by any technique capable of introducing a pharmacologically active agent to the desired site of action, including, but not limited to, buccal, sublingual, nasal, oral, topical, rectal and parenteral administration. Delivery of the pharmaceutical compositions may also be through the use of controlled release formulations in subcutaneous implants or transdermal patches.

For oral administration, a suitable pharmaceutical composition maybe prepared in the form of tablets, dragees, capsules, syrups and aqueous or oil suspensions. The inert ingredients used in the preparation of these compositions are known in the art. For example, tablets may be prepared by mixing the active compounds with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of at least one of the pharmacologically active agents. Such tablets may, if desired, be provided with enteric coatings by known methods, for example by the use of cellulose acetate phthalate. Suitable binding or granulating agents are, e.g., gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing the pharmacologically active agents and at least one diluent, and optionally a part of the disintegrating agent, is granulated together with an aqueous, ethanolic or aqueous-ethanolic solution of the binding agents in appropriate equipment, then the granulate is dried. Thereafter, other preservative, surface acting, dispersing, disintegrating, gliding and anti-adhesive additives can be mixed to the dried granulate and the mixture can be compressed to tablets or capsules.

The tablets may also be prepared by the direct compression of the mixture containing the pharmacologically active agents together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, a mixture of the active pharmaceutical agents and the desired additives may be filled into a capsule, such as a hard or soft gelatin capsule. The contents of a capsule and/or caplet may also be formulated using known methods to give sustained release of the active compound.

Liquid oral dosage forms of the active pharmaceutical agents may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing the pharmacologically active agents may be prepared in the form of a suppository. In addition to the pharmacologically active agents, the suppository may contain a suppository mass commonly used in pharmaceutical practice, such as Theobroma oil, glycerinated gelatin or a high molecular weight polyethylene glycol.

For parenteral administration, a suitable composition of the pharmacologically active agents may be prepared in the form of an injectable solution or suspension. For the preparation of injectable solutions or suspensions, the pharmacologically active agents can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspensions may be prepared from sterile powders or granules having one or more carriers or diluents mentioned for use in the formulations for oral administration. Parenteral administration may be through intravenous, intradermal, intramuscular or subcutaneous injections.

A composition containing the pharmacologically active agents may also be administered nasally, for example by sprays, aerosols, nebulized solutions and/or powders. Metered dose systems known to those in the art may also be used.

Pharmaceutical compositions of the pharmacologically active agents, may be administered to the buccal cavity (for example, sublingually) in known pharmaceutical forms for such administration, such as slow dissolving tablets, chewing gums, troches, lozenges, pastilles, gels, pastes, mouthwashes, rinses and/or powders.

Compositions containing the pharmacologically active agents for topical administration may comprise a matrix in which the pharmacologically active agents is dispersed such that it is held in contact with the skin in order to administer the agents transdermally. A suitable transdermal composition may be prepared by mixing the pharmacologically active agents with a topical vehicle, such as a mineral oil, petrolatum and/or a wax, for example paraffin wax or beeswax, together with a potential transdermal accelerant such as dimethyl sulphoxide or propylene glycol. Alternatively, the pharmacologically active compounds may be dispersed in a pharmaceutically acceptable cream or ointment base. The amount of the pharmacologically active agents contained in a topical formulation should be such that a therapeutically effective amount is delivered during the period of time for which the topical formulation is intended to be on the skin.

The pharmacologically active agents may also be administered by continuous infusion either from an external source, for example by intravenous infusion or from a source of the compound placed within the body. Internal sources include implanted reservoirs containing the pharmacologically active agents to be infused which is continuously released for example by osmosis and implants which may be (a) liquid such as a suspension or solution in a pharmaceutically acceptable oil of the compounds to be infused for example in the form of a very sparingly water-soluble derivative such as a dodecanoate salt or (b) solid in the form of an implanted support, for example of a synthetic resin or waxy material, for the compound to be infused. The support may be a single body containing all the compound or a series of several bodies each containing part of the compound to be delivered. The amount of the pharmacologically active agents present in an internal source should be such that a therapeutically effective amount is delivered over a long period of time.

In addition, an injectable solution the pharmacologically active agents can contain various additives including preservatives, such as benzyl alcohol, methyl or propyl 4-hydroxybenzoate, benzalkonium chloride, phenylmercury borate and the like; as well as antioxidants, such as ascorbic acid, tocopherol, sodium pyrosulfate and optionally complex forming agents, such as an ethylenediamine tetraacetate salt for binding any metal traces, as well as buffers for adjusting the pH value and optionally a local anaesthetizing agent, e.g., lidocaine. The injectable solution containing the pharmacologically active agents is filtered before filling into the ampule and sterilized after filling.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

### EXAMPLES

### Example 1

Memory Enhancment in Rats

The effects of the compositions of the invention on memory enhancement are tested on a T16 maze model. The T16 maze model is described in detail elsewhere (e.g. Shimada et al., European Journal of Pharmacology, Vol. 263, pp. 293-300 (1994); Spangler, Physiology & Behavior, Vol. 56, No. 1, pp. 95-101 (1994)).

Pretraining is conducted in a 2-meter long straight runway, constructed of clear Plexiglass. The floor is constructed of stainless steel grids wired to distribute a constant-current scrambled shock. A hand-held switch initiates a foot-shock and starts a clock that recorded the time to traverse the runway. Interchangeable black Plexiglas start and goal boxes can be placed over the grid floors at either end of the runway. On the day of pretraining, the rat is placed in one of the black boxes, the box is inserted into the start position of the runway, and a timer is initiated. The rat is pushed gently into the runway and is allowed 10 seconds to enter the goalbox to avoid a footshock (0.8 mA). Upon entry to the goal box, a guillotine door is lowered, the goal box is returned to the start area (after a 90 second ITI), and the next trial is initiated. Each rat continues to receive massed training trials until a criterion of 13/15 avoidances is met (max=30 trials).

Acquisition training is conducted in a clear Plexiglas 14-unit T-maze. The maze is separated into five distinct sections by guillotine doors that prevent animals from backtracking into previous sections. Nonfunctional guillotine doors at the entry to each cul-de-sac prevent the functional doors from being used as cues to the correct pathway. A switchbox triggers a clock which, when timed out, activates a counter that record the number of shocks (max=5/trial). Infrared photocells throughout the maze record the number of errors (defined as any deviation from the correct pathway) and runtime. The maze is surrounded by painted gray wooden walls to reduce extra-maze visual cues. Speakers are located under the maze at each corner and provide music to reduce auditory cues. The maze can be hoisted by motor driven pulleys to clean the grid floor and reduce the presence of odor cues.

For acquisition training, 24 hours after pretraining, the rat is placed into a start box and the box is inserted into the start position. The rat is pushed gently into the maze, the door is closed, and the clock controlling the shock contingency is initiated. In each section of the maze, the rat is given 10 seconds to escape through the door to the next section. After 10 seconds, a footshock is delivered until the rat escapes through the door. When the rat passes into the next section, the door is lowered behind the animal, and the clock contingency is reset. Upon entering the goal box, the door is closed, the box is placed in a holding area for 90 seconds, and the maze is hoisted for cleaning. Each rat receives a total of 15 massed trials with a 2-minute interial interview.

Rats are randomly assigned to treatment groups. Saline or a combination of galanthamine and simvastatin are administered by intraperitoneal injection approximately 30 minutes prior to initiation of testing. Doses are chosen to be within the therapeutically effective range for each compound.

### Example 2

Inhibition of Scopolamine-Induced Memory Impairment in Mice

The test is a test for the effect of a cognition enhancing substance on the latency of a mouse to move from an aversive white brightly illuminated compartment to a less aversive black dimly illuminated compartment on repeated exposure to the test situation and for the antagonizing effect of said substance on the memory impairing substance scopolamine in this test.

The test is conducted using an open-top experimental box (45×27×27 cm) two fifths of which was partitioned from the rest, painted black and illuminated with a dim red light (60 W). The remainder of the box is painted white and brightly illuminated (60 W) with a white light source. Access between the two compartments is by means of a 7.5×7.5 cm opening located in floor level at the center of the partition.

The mice are aged male albino (BKW) mice having an age 8-12 month (aged mice) housed in groups of 10 and given free access to drink and food and kept on a dark/light cycle of 12 hours. The test is carried out by placing the mice (taken from a dark home environment) in the center of the white section of the test box. The test period is 5 min. per day. The latency to move from the white to the black section is assessed via remote video recording. On day 4, scopolamine (0.25 mg/kg (control group of young mice) or 0.1 mg/kg (aged mice, test group and control group) i.p.b.d.) is given 40 min prior to testing. Test composition (galanthamine and simvastatin) is given i.p.b.d. before the testing. As control animals, young male albino (BKW) mice having an age of 6-8 month and a group of aged male albino (BKW) mice are used. Data obtained are analyzed by a one-way ANOVA followed by Dunnett's t-test.

What is claimed is:

1. A pharmaceutical composition for the treatment or prevention of memory impairment comprising:
(a) an effective amount of at least one cholinesterase inhibitor;
(b) an effective amount of at least one other pharmacologically active agent selected from the group consisting of HMG-CoA reductase inhibitors, cholesterol absorption inhibitors, gamma or beta secretase inhibitors, NMDA antagonists, muscarinic receptor agonists and nicotinic receptor agonists; and
(c) a pharmaceutically acceptable carrier.

2. The pharmaceutical composition according to claim 1, wherein said cholinesterase inhibitor is selected from the group consisting of physostigmine, tacrine and tacrine analogues, galanthamine, epigalanthamine, norgalanthamine, fasciculin, metrifonate, heptyl-physostigmine, norpyridostigmine, nomeostigmine, huperzine and pharmaceutically acceptable salts thereof.

3. The pharmaceutical composition according to claim 2, wherein said HMG-CoA reductase inhibitor is a statin.

4. The pharmaceutical composition according to claim 3, wherein said statin is selected from the group consisting of atorvastatin, simvastatin, pravastatin, lovastatin, fluvastatin, cerivastatin and pharmaceutically acceptable salts thereof.

5. The pharmaceutical composition according to claim 4, wherein said cholinesterase inhibitor is galanthamine or a pharmaceutically acceptable salt thereof.

6. The pharmaceutical composition according to claim 5, wherein said statin is simvastatin.

7. The pharmaceutical composition according to claim 1, wherein said cholesterol absorption inhibitor is ezetimibe.

8. The pharmaceutical composition according to claim 1, wherein said gamma or beta secretase inhibitor is selected from the group consisting of H-Lys-Thr-Glu-Glu-Ile-Ser-Glu-Val-Asn-Sta-Val-Ala-Glu-Phe-OH, presenilin-1 and presenilin-2.

9. The pharmaceutical composition according to claim 1, wherein said NMDA antagonist is selected from the group consisting of ketamine, phencyclidine, dizocilpine, tiletamine, dextromethorphan, memantine, amantadine, methadone, dextropropoxyphene, ketobemidone and pharmaceutically acceptable salts thereof.

10. The pharmaceutical composition according to claim 1, wherein the muscarinic receptor agonist is selected from the group consisting of aceclide, pilocarpine, oxotremorine, arecaidine, 5-methylfurmethiodide and pharmaceutically acceptable salts thereof.

11. The pharmaceutical composition according to claim 1, wherein the nicotinic receptor agonist is selected from the group consisting of nicotine, trans-metanicotine, epibatidine, pyridol, lobeline, imidacloprid and pharmaceutically acceptable salts thereof.

12. A method for treating or preventing memory impairment in a mammal in need thereof comprising administering to said mammal in need thereof:
(a) an effective amount of at least one cholinesterase inhibitor; and
(b) an effective amount of at least one other pharmacologically active agent selected from the group consisting of HMG-CoA reductase inhibitors, cholesterol absorption inhibitors, gamma or beta secretase inhibitors, NMDA antagonists, muscarinic receptor agonists and nicotinic receptor agonists.

13. The method according to claim 12, wherein said cholinesterase inhibitor is selected from the group consisting of physostigmine, tacrine and tacrine analogues, galanthamine, epigalanthamine, norgalanthamine, fasciculin, metrifonate, heptyl-physostigmine, norpyridostigmine, nomeostigmine, huperzine and pharmaceutically acceptable salts thereof.

14. The method according to claim 13, wherein said HMG-CoA reductase inhibitor is a statin.

15. The method according to claim 14, wherein said statin is selected from the group consisting of atorvastatin, simvastatin, pravastatin, lovastatin, fluvastatin, cerivastatin and pharmaceutically acceptable salts thereof.

16. The method according to claim 15, wherein said cholinesterase inhibitor is galanthamine or a pharmaceutically acceptable salt thereof.

17. The method according to claim 16, wherein said statin is simvastatin.

18. The method according to claim 12, wherein said mammal is a human.

19. The method according to claim 12, wherein said memory impairment is primary memory impairment.

20. The method according to claim 12, wherein said memory impairment is secondary memory impairment.

21. The method according to claim 12, wherein said memory impairment is associated with dementia.

22. The method according to claim 12, wherein said memory impairment is age associated memory impairment.

23. The method according to claim 12, wherein said at least one cholinesterase inhibitor and said at least one other pharmacologically active agent are administered substantially simultaneously.

24. The method according to claim 12, wherein said at least one cholinesterase inhibitor and said at least one other pharmacologically active agent are administered at spaced time intervals.
